(19) United States
(12) Patent Application Publication
(10) Pub. No.: US 2013/0211404 A1
(43) Pub. Date: Aug. 15, 2013

(54) SURGICAL TOOL FOR PECTUS BAR EXTRACTION

(71) Applicants: Frederic D. McKenzie, Virginia Beach, VA (US); Sebastian Bawab, Chesapeake, VA (US); Krzysztof Rechowicz, Norfolk, VA (US); Robert Obermeyer, Norfolk, VA (US)

(72) Inventors: Frederic D. McKenzie, Virginia Beach, VA (US); Sebastian Bawab, Chesapeake, VA (US); Krzysztof Rechowicz, Norfolk, VA (US); Robert Obermeyer, Norfolk, VA (US)

(21) Appl. No.: 13/840,145

(22) Filed: Mar. 15, 2013

Related U.S. Application Data
(63) Continuation-in-part of application No. 12/692,232, filed on Jan. 22, 2010.

(51) Int. Cl. A61B 17/92; A61B 17/88; A61B 17/68

(52) U.S. Cl. CPC A61B 17/92; A61B 17/68; A61B 17/88
USPC 606/60; 606/99

(57) ABSTRACT

A system for correcting pectus excavatum includes a pectus bar having an elongated main body portion with posterior and anterior sides and opposing ends, and a lateral cross section. A pectus bar extraction tool has a handle, the handle having a head at an end of the handle. The head has an elongated slot for receiving the pectus bar. The slot is dimensioned to receive the lateral cross section of pectus bar such that the pectus bar can be positioned in the slot and the handle manipulated to impart a force on the pectus bar. A pectus bar extraction tool and a method for removing a pectus bar are also disclosed.

### CROSS-REFERENCE TO RELATED APPLICATION

This Application is a Continuation-in-Part of U.S. Non-Provisional application Ser. No. 12/692,232, filed Jan. 22, 2010, entitled SURGICAL TOOL FOR PECTUS BAR EXTRACTION.

### BACKGROUND OF THE INVENTION

Pectus excavatum (PE), also called sunken or funnel chest, is a congenital chest wall deformity which is characterized, in most cases, by a deep depression of the sternum. Very often other problems can accompany this condition, like scoliosis and breathing issues. The correction of pectus excavatum (PE) and similar chest deformities has two main goals. Using the minimally-invasive Nuss procedure, one or two metal bars are placed to change the chest geometry which, in most cases, effects a positive result on a patient's physiology. Secondly, subjective aesthetic improvement may be achieved.

According to the Nuss procedure, incisions are made under the skin in the lateral chest wall at each lateral side of the patient and a skin tunnel is raised from the incision to the top of the pectus ridge. A tool known as a pectus introducer is inserted through the incision and used to create a tunnel for the pectus bar. The pectus introducer is advanced under the sternum and then through the opposite lateral incision. The pectus bar is shaped to fit the patient. The pectus bar is then inserted through the tunnel, with the convexity facing posteriorly. The pectus bar is then inverted to raise the sternum and anterior chest wall. The pectus bar is secured in place, for which one or more stabilizers can be used, and the incisions are closed.

The pectus bar remains in the patient for a treatment period lasting about 2 years or until the physician determines that the correction is complete and then is removed. Tissue grows around and in some cases directly adheres to the pectus bar, making removal difficult. Currently, surgical tools for removing the bars have proved inadequate for many surgeons.

### SUMMARY OF THE INVENTION

A pectus bar extraction tool for removing a pectus bar having posterior and anterior sides, includes a handle, an engagement member and a lever arm. The engagement member is fixed to and extends from the handle extending from the anterior to the posterior side of the pectus bar to engage the posterior side of the pectus bar. The lever arm is fixed to and extends from the handle opposite to the engagement member to rest upon the pectus bar on the anterior side of the pectus bar and act as a fulcrum.

The engagement member can provide a protrusion adapted for placement through an aperture in the pectus bar. The protrusion can be a hook. The width of the hook can be less than one-half of the width of the pectus bar. The hook extends distally to the handle.

The lever arm can comprise lateral side members for engaging lateral side portions of the pectus bar. A web can be provided between the lateral side members, and can be dimensioned so that the pectus bar will fit between the lateral side members and contact the web. The lateral side members can be rails.

The handle can include an indented portion. The indented portion assists in gripping when the handle is in proximity to a surface, such as an operating room table.

A system for correcting pectus excavatum can include a pectus bar and a pectus bar extraction tool. The pectus bar has an elongated main body portion with posterior and anterior sides and opposing ends, and structure at the ends for engaging the pectus bar extraction tool. The pectus bar extraction tool can include a handle and an engagement member fixed to the handle for cooperating with the engagement structure on the pectus bar to engage the extraction tool to the pectus bar. The engagement member of the pectus bar extraction tool can extend under the pectus bar to engage the posterior side of the pectus bar. The pectus bar extraction tool can have a lever portion for engaging the pectus bar on the anterior side.

A method for extracting a pectus bar from a patient includes the step of providing a pectus bar having an elongated main body portion with posterior and anterior sides and opposing ends, and engagement structure at the ends for engaging a pectus bar extraction tool, the pectus bar having been surgically implanted in the patient. A pectus bar extraction tool having a handle and an engagement member fixed to the handle for cooperating with the engagement structure on the pectus bar is also provided. The engagement member on the pectus bar extraction tool is engaged to the engagement structure on an opposing end of the pectus bar. A second pectus bar extraction tool can be engaged to the engagement structure at the other opposing end of the pectus bar. Using the handles, the extraction tools are manipulated to apply a straightening force to the pectus bar so as to at least partially straighten the pectus bar, whereby the pectus bar can be removed from the patient.

A system for correcting pectus excavatum includes a pectus bar having an elongated main body portion with posterior and anterior sides and opposing ends, and a lateral cross section. A pectus bar extraction tool includes a handle having a head at an end. The head includes an elongated slot for receiving the pectus bar. The slot is dimensioned to receive the lateral cross section of the pectus bar such that the pectus bar can be positioned in the slot and the handle manipulated to impart a force on the pectus bar.

The handle has a long axis and the elongated slot has a long axis. The long axis of the handle and the long axis of the slot can be substantially perpendicular.

The height of the slot can be no more than 2 mm greater than the height of the lateral cross section of the pectus bar, and the width of the slot can be no more than 2 mm greater than the width of the lateral cross section of the pectus bar. The height of the slot can be no more than 1 mm greater than the height of the lateral cross section of the pectus bar, and the width of the slot can be no more than 1 mm greater than the width of the lateral cross section of the pectus bar.

The pectus bar extraction tool can comprise an engagement member and the pectus bar can comprise structure at the opposing ends for engaging the engagement member. The engagement member of the pectus bar extraction tool when engaged to the pectus bar extends from the anterior to the posterior sides of the pectus bar to engage the posterior side of the pectus bar.

The head of the pectus bar extraction tool can further comprise a lever arm for engaging the pectus bar on the anterior side of the pectus bar. The engagement member and the lever arm can be aligned along an axis and extend oppositely from one another. The axis of the elongated slot can be transverse to the axis of the lever arm and engagement member. The engagement member can be a curved projection for extending from the anterior to the posterior sides of the pectus bar to engage the posterior side of the pectus bar. The projection can be a hook. The width of the hook can be less than one-half of the width of the pectus bar. The lever arm can comprise a step for contacting the pectus bar. The step can be provided at an end of the head and the hook can be provided at an opposite end of the head.

A pectus bar extraction tool for removing an elongated pectus bar from a patient, the pectus bar having anterior and posterior sides and a lateral cross section, the extraction tool comprising a handle having a head at an end of the handle, the head comprising an elongated slot for receiving the pectus bar, the slot being dimensioned to receive the lateral cross section of the pectus bar such that the pectus bar can be positioned in the slot.

The handle has a long axis and the elongated slot has a long axis, and the long axis of the handle and the long axis of the slot can be substantially perpendicular. The head of the pectus bar extraction tool can further comprise a lever arm for engaging the pectus bar on the anterior side of the pectus bar. The lever arm can include lateral side members for engaging lateral side portions of the pectus bar. The lateral side members can be rails.

A method for extracting a pectus bar from a patient can include the step of providing a pectus bar having an elongated main body portion with posterior and anterior sides and opposing ends, and a lateral cross section, where the pectus bar has been surgically implanted in the patient. A pectus bar extraction tool is provided. The pectus bar extraction tool comprises a handle. The handle has a head at an end of the handle. The head comprises an elongated slot for receiving the pectus bar. The slot is dimensioned to receive the lateral cross section of pectus bar such that the pectus bar can be positioned in the slot and the handle manipulated to impart a force on the pectus bar.

The pectus bar extraction tool is engaged to the pectus bar by positioning the pectus bar extraction tool adjacent an end of the pectus bar and moving the pectus bar extraction tool relative to the end of the pectus bar such that the end enters the slot of the pectus bar extraction tool. The pectus bar extraction tool is moved to a desired position on the pectus bar. The pectus bar extraction tool is then manipulated to apply a force to the pectus bar.

A second pectus bar extraction tool can be engaged to another opposing end of the pectus bar by positioning the other opposing end through an slot in the head of the second pectus bar extraction tool. The handles can then be used to apply forces from the first and second pectus bar extraction tools to the pectus bar.

The pectus bar extraction tool can further comprise an engagement member fixed to the head for cooperating with engagement structure on the pectus bar. The engagement member on the head comprises a projection and the engagement structure on the pectus bar comprises an aperture for receiving the projection. The projection on the pectus bar extraction tool is positioned in the aperture of the pectus bar. The handle is manipulated to apply a force from the projection to the pectus bar.

### BRIEF DESCRIPTION OF THE DRAWINGS

There is shown in the drawings embodiments which are presently preferred, it being understood, however, that the invention can be embodied in other forms without departing from the spirit or essential attributes thereof.

FIG. 1 is a side elevation of a pectus bar extraction tool according to the invention.

FIG. 2 is a perspective view of a distal end of the extraction tool.

FIG. 3 is a front elevation.

FIG. 4 is a cross section taken along line 4-4 in FIG. 3.

FIG. 5 is a cross section illustrating the extraction tool engaged to a pectus bar.

FIG. 6 is a cross section through the human anatomy illustrating the use of extraction tools according to the invention to remove a pectus bar according to a method of the invention, in a first mode of operation.

FIG. 7 is a cross section through the human anatomy illustrating the use of extraction tools to remove a pectus bar according to a method of the invention, in a second mode of operation.

FIG. 8 is a perspective view of a pectus bar extraction tool according to an alternative embodiment.

FIG. 9 is a rear elevation of an extraction tool head.

FIG. 10 is a cross-section taken along A-A in FIG. 9.

FIG. 11 is a side elevation.

FIG. 12 is a bottom view.

FIG. 13 is a perspective view, partially broken away, and showing the extraction tool engaged to a pectus bar.

FIG. 14 is a side elevation.

FIG. 15 is a cross-section taken along line A-A in FIG. 14.

FIG. 16 is a schematic diagram illustrating a use of the pectus bar extraction tool according to the alternative embodiment.

### DETAILED DESCRIPTION OF THE INVENTION

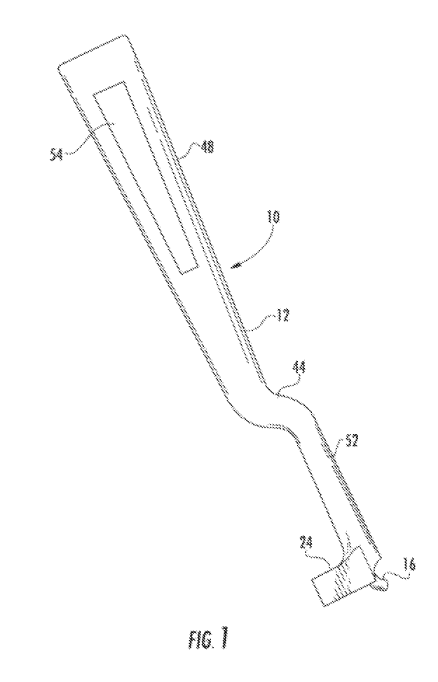

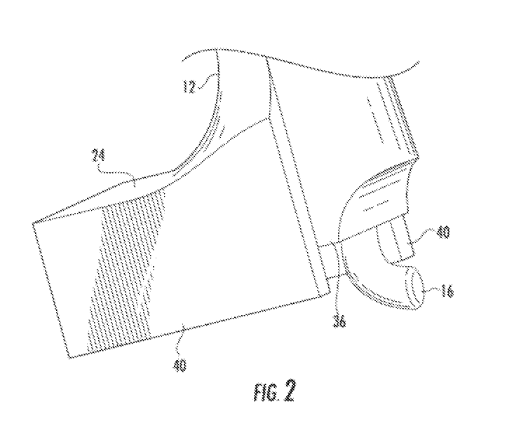

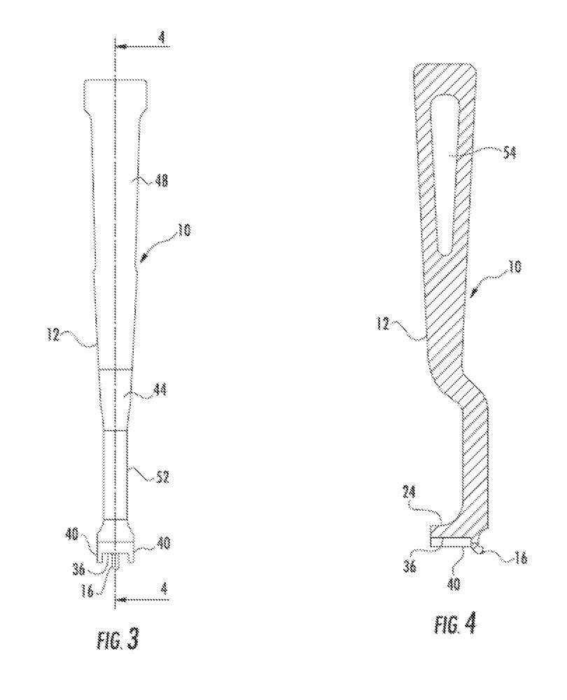

A pectus bar extraction tool according to the invention is shown in FIGS. 1-7. The extraction tool 10 according to the invention includes an elongated handle 12. An engagement member 16 is fixed to and extends from the handle 12. The engagement member 16 extends under a pectus bar 20 to engage the posterior side 32 of a pectus bar 20. A lever arm 24 is fixed to and extends from the handle 12 oppositely from the engagement member 16 for engaging the pectus bar 20 on the anterior side 28 of the pectus bar 20.

The engagement member 16 can be in the shape of a protrusion extending distally and outwardly relative to the handle 12 so as to provide a hook or claw shape. The dimensions of the engagement member can vary, and in one aspect the width of the hook is less than one-half the width of the pectus bar 20. The hook or other engagement member 16 can be provided with a rounded or blunt end so as to avoid damage to tissue during the pectus bar removal process. Other dimensions and configurations of the engagement member 16 are possible. A tongue and groove engagement, a flange and slot, hooks and loops, or various other detachable mechanical engagement structures are possible. The attachment should be secure enough to apply the forces required to straighten the pectus bar 20 and to free the pectus bar from surrounding tissue, and the attachment should be made from the anterior side 28 of the pectus bar 20 to the extent possible so that excessive cutting or tearing of tissue is not required to position the engagement member under or around the pectus bar 20.

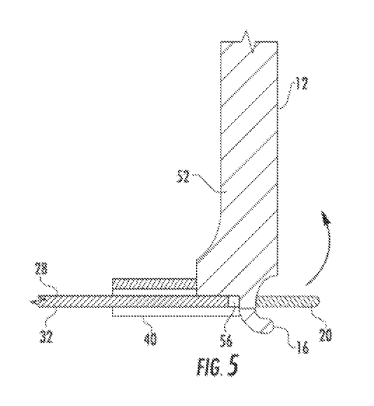

The lever arm 24 engages an anterior surface 28 of the pectus bar 20 while the engagement member 16 engages a posterior surface 32 (FIG. 5). The handle 12 can then be manipulated to rotate the engagement member 16 in the direction of the arrow, and the lever arm 24 will contact the anterior surface 28 so as to straighten the pectus bar 20 and to pry the pectus bar 20 from tissue to which it may be connected.

The lever arm 24 can comprise a web 36 and lateral side members such as rails 40. The web 36 provides a contact surface for the pectus bar 20 that is a distance from the handle 12 and engagement member 16 so as to act as a lever to lend additional force to the engagement member 16 and pry the pectus bar 20 from surrounding tissue. The lateral side members are positioned so as to receive the pectus bar 20 between them, and with the web 36, to provide for secure engagement of the pectus bar 20 so that the engagement member 16 does not become disengaged from the pectus bar 20 and is stabilized on the bar.

The handle 12 can be fashioned in different shapes and sizes. In one embodiment, the handle 12 has a curved portion 44 to offset the proximal portion 48 of the handle which is gripped by the user, from the distal portion 52. This will allow for gripping of the proximal portion 48 of the handle 12 when it is horizontal and adjacent to a surface such as an operating room table. An opening 54 can be provided in the proximal portion 48 to further facilitate gripping. The length of the handle can be adjusted for the preference of the user, the amount of leverage that is necessary, or the particular removal procedure that will be performed.

The invention can be used with pectus bars having a variety of shapes and sizes. The pectus bar 20 has at least one aperture 56. The engagement member 16 extends through the aperture 56 so as to engage the posterior surface 32. The pectus bar 20 is generally elongated and planar, and with differing dimensions according to the size and shape of the patient and the particular characteristics of the patient. The pectus bar 20 is usually made from a material that is pliable, so that the pectus bar 20 can be shaped in the operating room by the surgeon to fit the particular patient. The pectus bar 20 can be made from surgical quality metals or plastics.

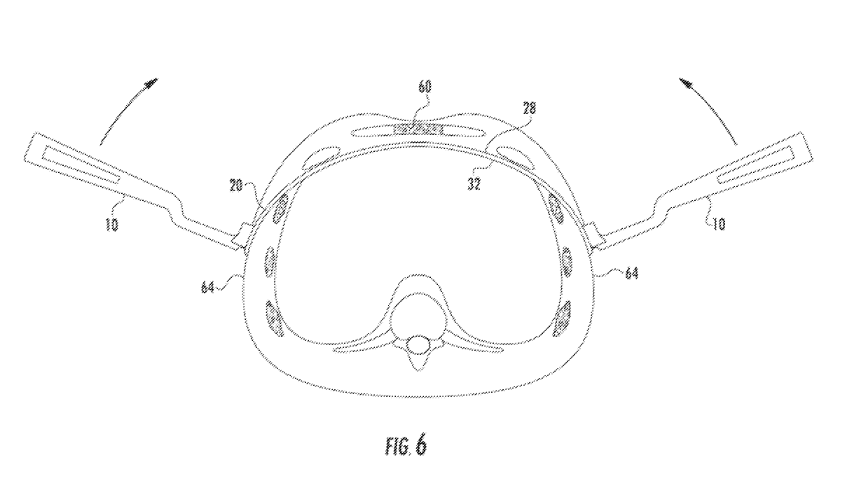

A method for extracting a pectus bar from a patient is shown in FIGS. 6-7. The engagement member 16 of the pectus bar extraction tool 10 is positioned through the aperture 56 and thereby engaged to the pectus bar 20. Preferably two extraction tools 10 are provided, one for each end of the pectus bar 20 at each lateral side 64 of the patient. The second pectus bar extraction tool is engaged to the engagement structure at the other end of the pectus bar 20. The handles of the pectus bar extraction tools 10 are used to apply a straightening force to the pectus bar so as to at least partially straighten the pectus bar 20, as depicted by the arrows in FIG. 6. A twisting motion can then be applied to the handles to assist in dislodging the pectus bar 20 from surrounding tissue, as depicted by the arrows in FIG. 7. The pectus bar 20 can then be removed from the patient from a position posterior to the sternum 60.

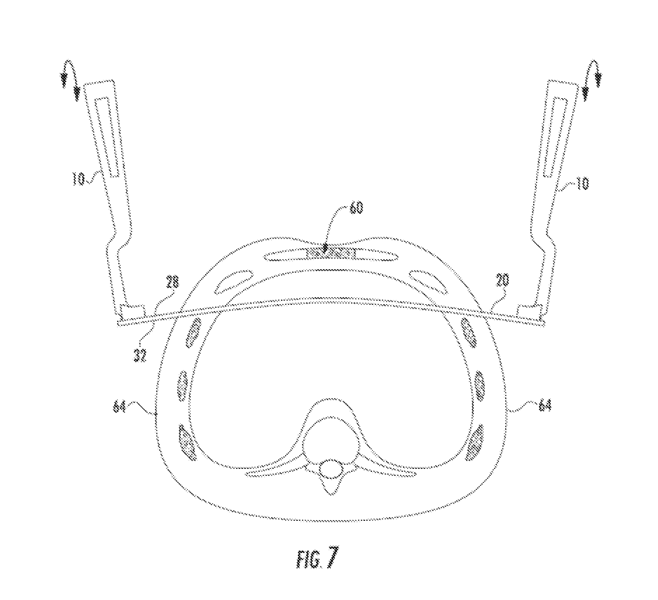

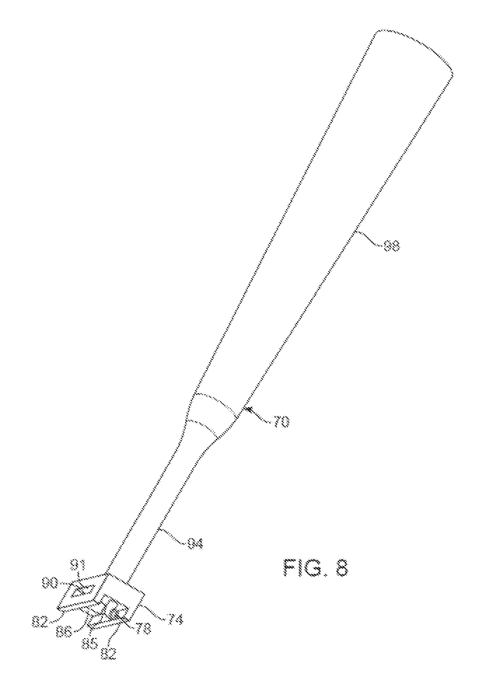

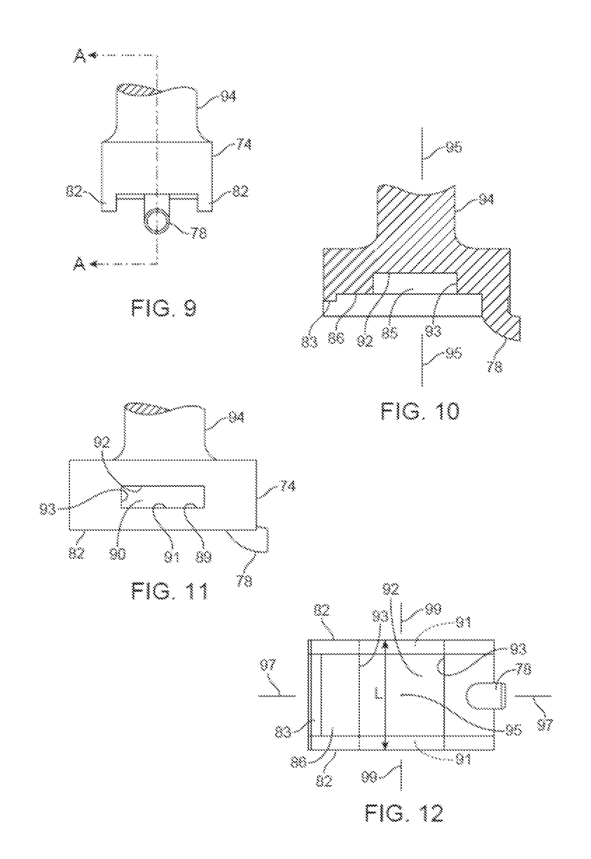

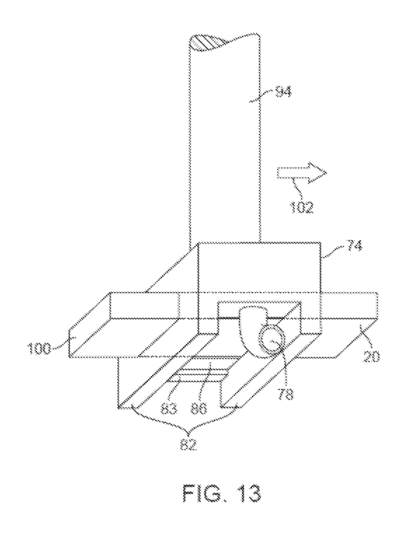

There is shown in FIGS. 8-16 an alternative pectus bar extraction tool 70. The extraction tool 70 has a head 74 communicating with a handle 94 with a long axis 95 (FIG. 10). The handle 94 can have a grip portion 98 to facilitate grasping and manipulation of the tool 70. A pectus bar engagement slot 90 is provided in the head 74. The slot 90 can be elongated and can have a long axis 99 extending through the slot 90 (see FIG. 12). The long axis 95 of the handle 94 and the long axis 99 of the elongated slot 90 can be substantially perpendicular. As used herein, substantially perpendicular means 90°, or 90°±20°, ±15°, ±10°, or ±5°. The slot 90 is dimensioned to receive the lateral cross-section of the pectus bar 20 such that the pectus bar 20 can be positioned in the slot 90 and the handle 94 manipulated to impart a force on the pectus bar 20. The height of the slot 90 is greater than the height of the lateral cross section of the pectus bar, and the width of the slot 90 is greater than the width of the lateral cross section of the pectus bar.

The slot 90 can be provided according to different constructions of the head 74. In the embodiment shown, the slot 90 comprises an opening 91 on each lateral side of the head 74. An open-faced groove 85 formed by top wall 92 and side walls 93 is fashioned in the head 74 between the openings 91 and is dimensioned to receive the pectus bar 20. The groove 85 adjoins the openings 91, such that the top wall 92 and side walls 93 of the groove 85 also form the top and side walls of the openings 91. The opening 91 has a bottom wall 89. It is alternatively possible to fashion a fully-enclosed slot 90 in the head 74. Other constructions for the slot 90 are possible.

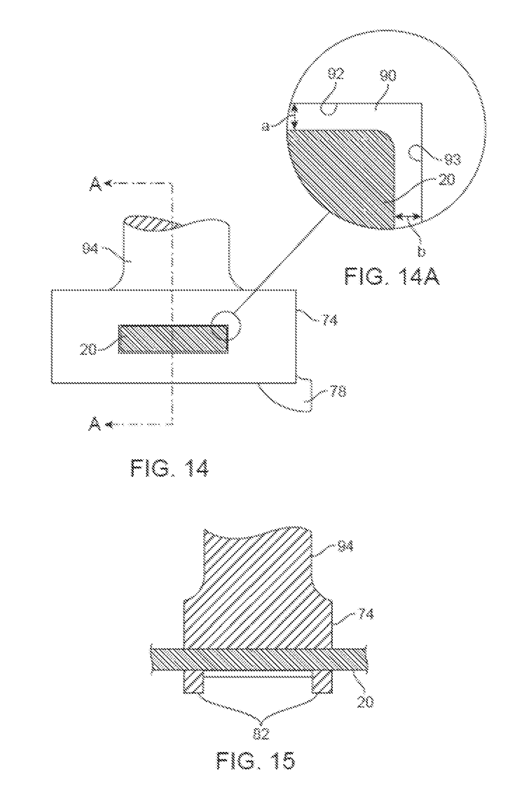

The height and width of the slot 90, and/or the openings 91 forming a part of the slot 90, are selected relative to the cross-sectional dimensions of the pectus bar 20 such that a significant amount of play between the tool 70 and the pectus bar 20 is not possible. The dimensions of the slot 90 are preferably just wide enough receive the pectus bar 20. In one aspect, the height and width dimensions of the slot 90 are each no more than 1 mm greater than the height and width dimensions of the pectus bar 20. This results in a gap (distances a and b in FIG. 14A) of no more than 1 mm. In another aspect the height and width of the slot 90 are no more than 0.5 mm to 2 mm greater than the height and width of the pectus bar 20, or any length there between. In one example, the dimensions of the lateral cross-section of the pectus bar 20 is 2.9 mm in height and a 12.7 mm in width, and the dimensions of the slot 90 are about 3.9 mm in height and 13.7 mm in width. This will provide small gaps a and b between the pectus bar 20 and the walls 92, 93 of the slot 90 so that the extraction tool 70 can slip easily over the pectus bar 20, but without significant play between the pectus bar 20 and the walls 92, 93 of the slot 90. The tool 70 may thereby be used to manipulate the pectus bar 20 without undue wobble or play. The desired difference in dimensions between the slot 90 and the lateral cross sectional dimensions of the pectus bar 20 can vary, for example, for pectus bars having greater dimensions the gap distances a and b can be greater, while for pectus bars having smaller distances the gap distances can be less. The gap distances a and b should be greater than 0 but no more than 1% to 30% of the respective height or width dimension of the pectus bar 20, or any percentage there between, such as 5%, 10%, 15%, 20%, or 25%.

The length L of the slot 90 (FIG. 12) is preferably no more than 2 times the length of the head 74 as measured along the axis 97. The length L will be limited by the dimensions of the head 74. The length L should not be too small or a bending force will be applied to the pectus bar 20 when the extraction tool is used to apply a force to the pectus bar 20. The length L should not be too long, as a slot 90 that is too long will be difficult to position over a curved section of the pectus bar 20.

As can be seen in FIGS. 13-15, the head 74 can be positioned over the pectus bar 20 by positioning the tool 70 such that the end 100 of the pectus bar 20 enters the slot 90. The tool 70 is then moved laterally inward from the end 100 in the direction indicated by the arrow 102 such that tool 70 engages the pectus bar 20 (FIG. 13).

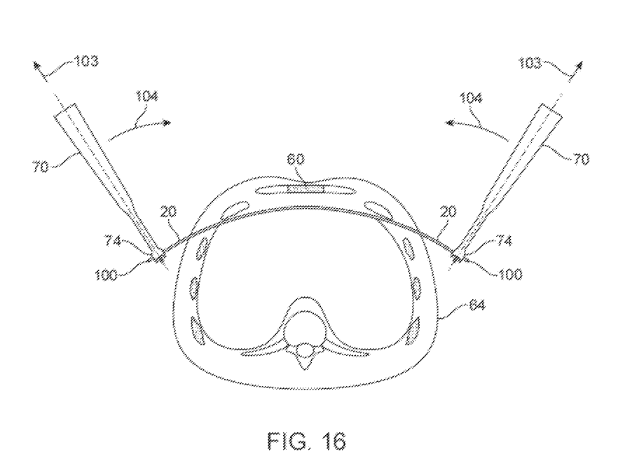

The operation of the alternative extraction tool 70 is shown in FIG. 16. The extraction tool 70 is positioned laterally inward from the end 100 of the pectus bar 20. The extraction tool 70 is then used to lift and pry the pectus bar 20, as indicated by the arrows 103-104. The pectus bar 20 will be securely engaged within the slot 90 such that slippage and wobble are minimized. A second extraction tool 70 can be positioned over the opposite end 100 of the pectus bar 20. The two tools 70 can then be manipulated in the fashion shown in FIG. 16 to manipulate the pectus bar 20.

The alternative extraction tool 70 can have additional structure to assist in removal of the pectus bar 20. In one embodiment, the head 74 has an engagement member 78 as previously described fixed to and extending from the head 74. The head 74 can also have a lever arm 86 and lateral side rails 82. A protruding flange or step 83 can be provided to engage the pectus bar 20 in a downward and countervailing direction to the upward force imparted by the engagement member 78. The engagement member 78 and lever arm 86 can be aligned along an axis 97 (FIG. 12) and extend oppositely to one another relative to the axis 95 of the handle 94. The axis 99 of the slot 90 can be transverse to, and can be substantially perpendicular to, the axis 97. As used herein, substantially perpendicular means 90°, or 90°±20°, ±15°, ±10°, or ±5°. As previously described the pectus bar 20 can fit between the rails 82 to assist in aligning the engagement tool 70 and to prevent twisting. The engagement member 78 extends through an aperture in the pectus bar 20 and to an opposite side of the pectus bar 20 to engage the pectus bar 20. The handle 94 can thereby be manipulated to force the lever arm 86 against the pectus bar 20, while prying upward against the anterior side of the pectus bar 20 using the engagement member 78, as shown in FIG. 5.

The engagement member 78 is first used to lift the ends 100 of the pectus bar 20 from the tissue of the patient. The engagement member 78 is engaged to the pectus bar 20 by placing the engagement member 78 through the aperture 56, whereby the pectus bar 20 can be pried and lifted from the tissue in which the pectus bar 20 has been embedded during the remodeling process. The engagement member 78 can be a protrusion or a hook. The diameter and material of the hook 78 can be adapted for the stresses that will be applied to the hook 78. The diameter of the hook should be as large as will permit insertion through the aperture 56 of the pectus bar 20 without collision. A high strength metal such as 17-4 H900 stainless steel can be used to provide additional strength to the hook 78. Lifting the pectus bar 20 out of the tissue enables the end 100 of the pectus bar 20 to be positioned in the slot 90. The tool 74 can then be moved to any desired position on the pectus bar 20 to impart lifting and prying forces wherever desired.

The length of the head 74 along the axis 97 (FIG. 12) can vary. Increasing this length will decrease the forces acting on the hook 78. The longer the head 74, the longer the incision must be on the side of the patient. This incision length is typically about 30 mm to 40 mm. In one aspect this length can be from 20 mm to 30 mm, and in another aspect can be 22 mm to 27 mm.

The step 83 can be provided at an end of the head 74 that is opposite to the end having the hook 78 so as to insure that the distance from the point of contact between the pectus bar 20 and the step 83 of the lever arm 86 is as far as possible from the point of contact of the hook 78 with the pectus bar 20. This will act to decrease the forces acting on the hook 78.

This invention can be embodied in other forms without departing from the spirit or essential attributes thereof and, accordingly, reference should be had to the following claims rather than the foregoing specification as indicating the scope of the invention.

We claim:

1. A system for correcting pectus excavatum, comprising:
a pectus bar having an elongated main body portion with posterior and anterior sides and opposing ends, and a lateral cross section;
a pectus bar extraction tool comprising a handle, the handle having a head at an end of the handle, the head comprising an elongated slot for receiving the pectus bar, the slot being dimensioned to receive the lateral cross section of the pectus bar such that the pectus bar can be positioned in the slot and the handle manipulated to impart a force on the pectus bar.

2. The pectus bar extraction tool of claim 1, wherein the handle has a long axis and the elongated slot has a long axis, and the long axis of the handle and the long axis of the slot are substantially perpendicular.

3. The pectus bar extraction tool of claim 1, wherein the lateral cross section of the pectus bar has a height and a width and the slot has a height and a width, the height of the slot being no more than 2 mm greater than the height of the pectus bar, and the width of the slot is no more than 2 mm greater than the width of the pectus bar.

4. The pectus bar extraction tool of claim 1, wherein the lateral cross section of the pectus bar has a height and a width and the slot has a height and a width, the height of the slot being no more than 1 mm greater than the height of the pectus bar, and the width of the slot is no more than 1 mm greater than the width of the pectus bar.

5. The system of claim 1, wherein the pectus bar extraction tool comprises an engagement member and the pectus bar comprises structure at the opposing ends for engaging the engagement member.

6. The system of claim 5, wherein said engagement member of the pectus bar extraction tool when engaged to the pectus bar extends from the anterior to the posterior sides of the pectus bar to engage the posterior side of the pectus bar.

7. The system of claim 6, wherein the head of the pectus bar extraction tool further comprises a lever arm for engaging the pectus bar on the anterior side of the pectus bar.

8. The system of claim 7, wherein the engagement member and the lever arm are aligned along an axis and extend oppositely from one another.

9. The system of claim 8, wherein the axis of the elongated slot is transverse to the axis of the lever arm and engagement member.

10. The system of claim 9, wherein the engagement member is a curved projection for extending from the anterior to the posterior sides of the pectus bar to engage the posterior side of the pectus bar

11. The system of claim 10, wherein the projection is a hook.

12. The system of claim 11, wherein the hook is provided at an end of the head, and the lever arm comprises a step, the step being provided of the lever arm at an end of the head opposite to the end having the hook.

13. A pectus bar extraction tool for removing an elongated pectus bar from a patient, the pectus bar having anterior and posterior sides and a lateral cross section, the extraction tool comprising a handle having a head at an end of the handle, the head comprising an elongated slot for receiving the pectus bar, the slot being dimensioned to receive the lateral cross section of the pectus bar such that the pectus bar can be positioned in the slot.

14. The pectus bar extraction tool of claim 13, wherein the handle has a long axis and the elongated slot has a long axis, and the long axis of the handle and the long axis of the slot are substantially perpendicular.

15. The pectus bar extraction tool of claim 13, wherein the lateral cross section of the pectus bar has a height and a width and the slot has a height and a width, the height of the slot being no more than 2 mm greater than the height of the pectus bar, and the width of the slot is no more than 2 mm greater than the width of the pectus bar.

16. The pectus bar extraction tool of claim 13, wherein the lateral cross section of the pectus bar has a height and a width and the slot has a height and a width, the height of the slot being no more than 1 mm greater than the height of the pectus bar, and the width of the slot is no more than 1 mm greater than the width of the pectus bar.

17. The pectus bar extraction tool of claim 13, wherein the head comprises an engagement member fixed to and extending from the head for engaging an opening in the pectus bar.

18. The pectus bar extraction tool of claim 17, wherein the engagement member is a curved projection for extending from the anterior to the posterior sides of the pectus bar to engage the posterior side of the pectus bar.

19. The pectus bar extraction tool of claim 18, wherein the projection is a hook.

20. The pectus bar extraction tool of claim 19, wherein the hook extends distally to the handle.

21. The pectus bar extraction tool of claim 20, wherein the head comprises a lever arm for engaging the pectus bar on the anterior side of the pectus bar, and wherein the hook and the lever arm are aligned along an axis and extend from the axis of the handle oppositely from one another.

22. The pectus bar extraction tool of claim 21, wherein the lever arm comprises a step for contacting the pectus bar, the step being provided at an end of the head, and the hook being provided at an end of the head that is opposite to the end having the step.

23. The pectus bar extraction tool of claim 22, wherein the axis of the elongated slot is transverse to the axis of the lever arm and the hook.

24. The pectus bar extraction tool of claim 13, wherein the head comprises a lever arm for engaging the pectus bar on an anterior side of the pectus bar.

25. The pectus bar extraction tool of claim 24, wherein the lever arm comprises lateral side members for engaging lateral side portions of the pectus bar.

26. The pectus bar extraction tool of claim 25, wherein the lateral side members are rails.

27. A method for extracting a pectus bar from a patient, comprising the steps of:
providing a pectus bar having an elongated main body portion with posterior and anterior sides and opposing ends, and a lateral cross section, the pectus bar having been surgically implanted in the patient;
providing a pectus bar extraction tool comprising a handle, the handle having a head at an end of the handle, the head comprising an elongated slot for receiving the pectus bar, the slot being dimensioned to receive the lateral cross section of pectus bar such that the pectus bar can be positioned in the slot and the handle manipulated to impart a force on the pectus bar;
engaging the pectus bar extraction tool to the pectus bar by positioning the pectus bar extraction tool adjacent an end of the pectus bar and moving the pectus bar extraction tool relative to the end of the pectus bar such that the end enters the slot of the pectus bar extraction tool;
moving the pectus bar extraction tool to a desired position on the pectus bar; and,
manipulating the pectus bar extraction tool to apply a force to the pectus bar.

28. The method of claim 27, further comprising the steps of engaging a second pectus bar extraction tool to another opposing end of the pectus bar by positioning the other opposing end through an slot in the head of the second pectus bar extraction tool; and,
using the handles to apply forces from the first and second pectus bar extraction tools to the pectus bar.

29. The method of claim 28, wherein the pectus bar extraction tool further comprises an engagement member fixed to the head for cooperating with engagement structure on the pectus bar, the engagement member on the head comprising a projection and the engagement structure on the pectus bar comprising an aperture for receiving the projection;
positioning the projection on the pectus bar extraction tool in the aperture of the pectus bar; and,
manipulating the handle to apply a force from the projection to the pectus bar.
